(19) United States
(12) Patent Application Publication
(10) Pub. No.: US 2007/0197937 A1
(43) Pub. Date: Aug. 23, 2007

(54) Microfluid system and method for production thereof

(76) Inventors: Emad Sarofim, Hagendorn (CH); Irio Giuseppe Calasso, Arth (CH); Patrick Griss, Otelfingen (CH)

Correspondence Address: ROCHE DIAGNOSTICS OPERATIONS INC., 9115 Hague Road, Indianapolis, IN, 46250-0457, US

(21) Appl. No.: 11/676,398

(22) Filed: Feb. 19, 2007

Related U.S. Application Data
(63) Continuation of application No. PCT/EP05/08934, filed on Aug. 18, 2005.

(30) Foreign Application Priority Data
Aug. 20, 2004 (EP) 04019759.2

(51) Int. Cl. A61B 5/00

(52) U.S. Cl. 600/583; 600/573

(57) ABSTRACT

The invention generally concerns a microfluidic system having a support body provided with a lancing member and a semi-open microchannel, for the capillary transport of a sample fluid from a receiving site to a target site. In order to obtain a higher aspect ratio the support body is coated with a build-up layer which laterally defines the microchannel in the upper region.

### REFERENCE TO RELATED APPLICATIONS

The present application is a continuation of PCT Patent Application No. PCT/EP2005/008934, filed Aug. 18, 2005 which claims priority to European Patent Application No. 04019759.2, filed Aug. 20, 2004, which are hereby incorporated by reference in their entirety.

### TECHNICAL FIELD

The invention generally concerns a microfluidic system. In particular to a microfluidic body having a support body provided with a lancing member and a semi-open microchannel.

### BACKGROUND

Microfluidic systems typically allow, especially in bioanalytics, the analysis of very small amounts of fluid. For example, such systems can be used to analyze fluids taken in situ as capillary blood for blood glucose determinations. In addition to the microscopic volumes (microliters and less) microfluidics are characterized by structural elements that have smaller dimensions which allow capillary forces to be utilized. In additional, the smaller dimensions have to be implemented in so-called disposables in a manner that is cost effective and suitable for mass production. Although such processes are known from the field of semi-conductor technology in the form of photochemical etching for highly integrated systems, the materials used for this purpose can hardly be used for mechanically stressed structures due to their brittleness. When biocompatible materials such as steel are etched, the problem occurs that the cross-sections of the generated channel structures do not allow optimal liquid transport due to the isotropic loss of material.

Prior art methods of manufacturing microfluidic also include application of a welling agent to the surface to increase fluid transportation. However, the prior techniques are less desirable since additional production steps are necessary.

Typically compatibility with a detection method for an analyte in the transported sample is required (i.e. no effect on the measurement result or no unacceptable falsification of the measurement result). It also has to be biocompatible (no toxic effects whatsoever) since when samples are taken it is not possible to rule out that parts coated with the wetting agent briefly penetrate the organism. In addition, The hydrophilization must have an adequate storage stability.

There are physical limitations when a wetting agent is used alone without a suitable geometry. Such limitations are individually or in combination due to the required transport distance, independence of position/gravitation and/or flow rate.

Therefore, there is a need to avoid the disadvantages that occur in the prior art and for an improved microfluidic system and a production process such that structures are created for an effective transport of small amounts of fluids using advantageous measures.

### SUMMARY

In accordance with the first aspect of the invention, the support body of a microfluidic system is coated with a build-up layer which laterally defines the microchannel at least in the upper region. The coating allows a firmly adhering structure to be formed in a simple manner with a previously shapeless substance whereby the channel formation or heightening in the build-up layer or on the side walls thereof results in a liquid-conducting fluidic function which is based on an increase in the capillarity. This means that channel cross-sections with a high aspect ratio which decisively improve the capillary action can also be formed on isotropically etchable substrates. The support body can at the same time be designed as a lancing element for lancing the skin or alternatively can have a collecting or receiving function that is separate from a lancing element.

In yet another aspect of the invention, the microchannel has a lower cross-sectional region that is etched into the support body and an overlying upper cross-sectional region formed in the build-up layer. It is also possible that the build-up layer laterally delimits the microchannel over its entire depth and thus alone has a liquid-conducting function.

In yet another aspect of the invention, the build-up layer consists of a photoresist. This allows microfluidic structures which have the required rigidity and inertness for the end use to be formed on a support in a simple manner. This can be achieved by means of the fact that the build-up layer is photostructured in order to form or increase the height of the microchannel such that even complex geometries can be created with the required accuracy. The build-up layer has a layer thickness of more than 50 μm, typically in the range of 200 to 500 μm.

Yet another aspect of the invention is that the microchannel has several partial cross-sections etched down into the support body by successive etching steps starting from one surface of the support body. This also enables a large ratio of depth to width of the microchannel to be achieved in an isotropically etchable support material. The aspect ratio achieved by this method is longer than 0.5. The microchannel has an inner width in the range of 50 to 500 μm.

In yet another aspect of the invention, the partial cross-sections in the support body are formed by photochemical mask etching.

In yet another aspect of the invention, capillarity of the microchannel can also be increased by providing an undercut in the region of its longitudinal edges that one formed by underetching.

In yet another aspect of the invention, the support body consists of an isotropically etchable material. In yet another aspect, the support body has a flat shaped part made of metal such as a high-grade steel that will improve handling rigidity, inertness and biocompatibility of the microfluidic system. The support body formed from a flat material has a thickness of 100 to 450 μm.

In yet another aspect of the invention, the build-up layer has an additional substance or composition which increases the hydrophilicity. In yet another aspect of the invention, the wettability of a wall of the microchannel is increased by a chemical surface treatment.

In yet another aspect of the invention, the lancing member is formed outside of the microchannel region by etching or punching so that the various structures are created by uniform processes.

In yet another aspect of the system, the microfluidic system is used to transport a sample liquid from a receiving site to a target site such as a detection region for detecting the concentration of an analyte in the sample liquid.

In yet another aspect, the process of manufacturing a system having a microchannel is achieved by applying a photoresist layer to a support body to increase the height of or to form a microchannel which transports liquid.

In yet another aspect, the microchannel is etched into the support body by mask etching a first photoresist layer and, after removing the first photoresist layer, applying a second photoresist layer which is photostructured in order to increase the height of the microchannel.

These and other features and advantages of the present invention will be more fully understoof from the following detailed description of the invention taken together with the accompanying claims. It is noted that the scope of the claims is definitely by the recitations therein and not by the specific discussion of the features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a sample collection element as a microfluidic system to transport a sample liquid in a perspective view.

FIGS. 2 to 4 show the system according to FIG. 1 with a different build-up layer of a microchannel in cross-section.

FIGS. 5a to f show successive process steps for increasing the height of the channel by photostructuring the system according to FIG. 1 in cross-section, and

FIGS. 6a to k show successive process steps for deepening the channel in a view corresponding to FIG. 5.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

In order that the invention may be more readily understood, reference is made to the following examples, which are intended to illustrate the invention, but not limit the scope thereof.

### DETAILED DESCRIPTION

The following description of the preferred embodiment is merely exemplary in nature and is in no way intended to limit the invention or its application or uses.

The microfluidic system shown in the drawing as a disposable sample collection element 10 that enables the collection and capillary transport of small amounts of body fluid. Referring in particular to FIG. 1, the system 10 comprises a flat support body 12, a lancing member 14 formed thereon and a capillary microchannel 16 which at least in certain areas can be delimited by a build-up layer 18 of the support body 12.

The support body 12 as a strip-shaped flat formed part consists of steel of a thickness of about 150 to 300 μm. Its proximal end section forms a holding region 20 in order to handle it during the lancing process whereas the lancing member 14 moulded as one piece on the distal end generates a small wound in the skin of the user in order to be able to collect microscopic volumes of blood or tissue fluid.

The length of the microchannel 16 is shaped like a groove or is semi-open so that it is possible to manufacture it by photolithography as described in the following. Liquid can be effectively taken up from the skin or from the skin surface at the receiving site 22 in the region of the lancing member (lancet tip 14) via the semi-open cross-section without parts of tissue being able to completely close the entrance cross-section as is the case for conventional hollow cannulas.

Liquid is transported through the capillary channel 16 to the target site 24 which is at a distance from the lancing member 14 and at which the body fluid can be analyzed. The analysis of the body fluid can be achieved in a known manner by reflection spectroscopic or electrochemical detection methods.

The channel cross-section can be constant or can vary over the length of the microchannel 16. The width of the channel is in the range of 50 to 500 μm, whereas the so-called aspect ratio between depth and width is larger than 0.5 and larger than 0.8 to improve the capillarity of the microchannel. In this connection care should be taken that an approximately semi-circular cross-section is obtained with an aspect ratio of only 0.5 when the channel 16 is isotropically etched into the support body 12.

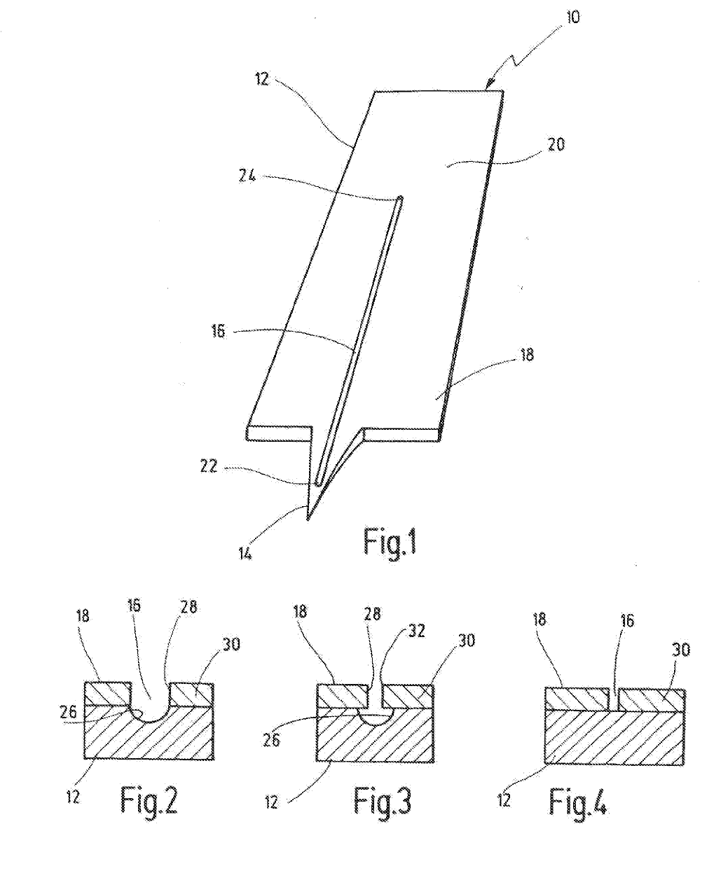

As shown in FIG. 2, the semi-circular lower channel region 26 formed by isotropic etching and acting as a bottom region in the support body or substrate 12 can be increased in height by the build-up layer 18 while laterally delimiting an upper open-edged channel region 28 thus obtaining overall a higher aspect ratio and hence a better capillary action for liquid transport. For this purpose the build-up layer 18 should have a layer thickness of more than 5 μm, and in the range of 200 to 500 μm.

The build-up layer 18 is not laminated as a prefabricated body onto the support body 12 but is applied as a permanently adhering layer from a previously shapeless substance. A coating material is intended for this purpose and in particular a photoresist 30. A thick film photoresist for example based on epoxy is suitable.

In the embodiment according to FIG. 2, the photoresist 30 is applied subsequently after etching the lower region 26 such that the complementary upper channel region 28 can additionally convey liquid. For this purpose the hydrophilicity of the layer 18 is increased by suitable additives or by an appropriate lacquer composition. It is also possible to improve the water affinity of the channel walls by a chemical surface treatment after structuring.

In the embodiment according to FIG. 3, the photoresist 30 used as a mask for etching the lower region 26 on the support body 12 is not removed but is retained for an additional fluidic function. As shown in addition to increasing the height of the channel walls it is also possible to reduce the surface 32 that is open towards the atmosphere by the undercut which further increases the capillarity. It is basically also conceivable to manufacture an undercut edge region of the channel 16 as an underetched structure of the support body 12 by suitable selection of the etching parameters.

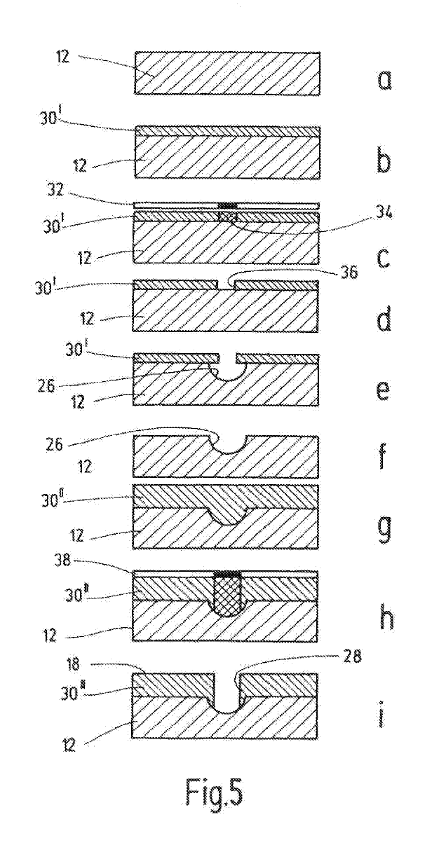

FIG. 4 shows an embodiment in which the build-up layer 18 laterally delimits the microchannel 16 over its entire depth wherein in this case it is also possible to achieve a high aspect ratio by an appropriate layer thickness of the photoresist 30. In addition to the photostructuring of the channel 16 in the layer 18, the support body can be structured by prior (isotropic) etching for example by etching out the lancing member 14.

FIG. 5 illustrates a process sequence for photostructuring the channel 16 on a previously etched support structure. Firstly the support body 12 as a substrate is provided with a first photoresist layer 30′ (FIG. 5a,b). This is followed by a UV exposure through the photomask 32 whereupon The photoresist 30′ is polymerized or hardened under the light-permeable regions of the mask whereas the masked regions 34 are rinsed clear after exposure and development (FIG. 5c,d). Subsequently an etching agent is applied to the support body 12 over the cutout 36 thus generated in the layer 30′ to isotropically etch out the channel region 26. After removing the photoresist layer 30′ (FIG. 5f) a channel elevation 28 is formed by further photostructuring of a second thick film layer 30″ using mask 38 according to the already pre-etched channel course (FIG. 5i). The hardened photoresist remains permanently on the substrate 12 as a build-up layer 18 and thus fulfills a fluidic function for an improved liquid transport.

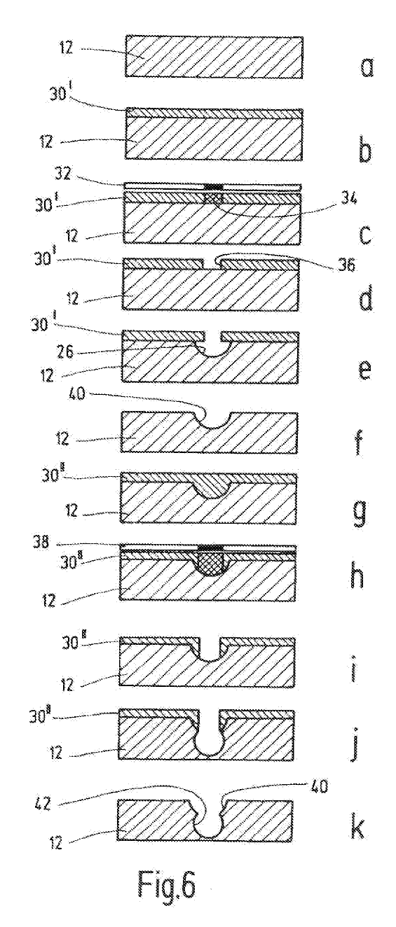

In the process sequence shown in FIG. 6, the aspect ratio of the channel 16 is increased by several successive etching steps. An upper partial cross-section 40 of the channel 16 is formed in the support body 12 by a first etching according to the previous description of FIGS. 5a to f (FIG. 6a to f). Then a deepened partial cross-section 42 is generated by repeating these steps at least once in a second or further etching so that channel 16 penetrates almost the entire support body 12 without extending isotropically in width (FIG. 6g to k). It is basically possible to carry out the etchings in parallel in opposing directions from both sides of the support body 12 until the channel 16 has been completely etched through in which case at least the bottom side must be closed for example by laminating on a foil.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent he inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modification and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A microfluidic system for collecting body fluids, the system Comprising:
a support body comprising a lancing member;
a semi-open microchannel, located on the support body such that the microchannel assists in the capillary transport of a fluid from a receiving site to a target site; and
a build-up layer coated on the support body for the fluid transport which laterally defines the microchannel in the upper region.

2. The system according to claim 1, wherein the microchannel has a lower cross-sectional region that is etched into the support body and an overlying upper cross-sectional region formed in the build-up layer.

3. The system according to claim 1, wherein the build-up layer laterally delimits the microchannel over its entire depth.

4. The system according to claim 1, wherein the build-up layer consists of a thick film photoresist.

5. The system according to claim 1, wherein the build-up layer is photostructured in order to form or increase the height of the microchannel.

6. The system according to claim 1, wherein the build-up layer has a layer thickness of more than 50 μm.

7. The system according to claim 6, wherein the thickness of the build-up layer is 200 to 500 μm.

8. The system according to claim 1, wherein the microchannel has several partial cross-sections etched down into the support body by successive etching steps starting from one surface of the support body.

9. The system according to claim 8, wherein the partial cross-sections are formed by photochemical mask etching.

10. The system according to claim 1, wherein a aspect ratio depth to width of the microchannel is larger than 0.5.

11. The system according to claim 1, wherein the microchannel has an inner width in the range of 50 to 500 μm.

12. The system according to claim 1, wherein the microchannel has an undercut in the region of its longitudinal edges formed by underetching.

13. The system according to claim 1, wherein the support body consists of an isotropically etchable material.

14. The system according to claim 1, wherein the support body has a flat shaped part consisting of metal.

15. The system according to claim 14, wherein the support body formed from a flat material has a thickness of 100 to 450 μm.

16. The system according to claim 1, wherein the build-up layer has an additional substance or composition which increases the hydrophilicity.

17. The system according to claim 1, wherein the wettability of a wall of the microchannel is increased by a chemical surface treatment.

18. The system according to claim 1, wherein the lancing member is formed by etching or punching and is positioned outside the microchannel.

19. A method of manufacturing a microfluidic system to collect a body fluid, the method comprising:
providing a support body having a lancing member; and
applying a photoresist layer to the support body to form a semi-open microchannel that transports body fluid from a receiving site to a target site.

20. The method according to claim 19, wherein the photoresist is sprayed or knife coated onto the support body as a thick film or is applied by dip coating.

21. The method according to claim 19, wherein the process of forming the microchannel comprises the steps of:
photochemically etching a first photoresist layer;
removing the first photoresist layer;
applying a second photoresist layer and its photostructured in order to increase the height of the microchannel.
